(19) United States
(12) Patent Application Publication
(10) Pub. No.: US 2025/0078992 A1
(43) Pub. Date: Mar. 6, 2025

(54) HIGH INTELLIGENCE VERTICAL EFFICIENCIES (HIVE) SYSTEM FOR TREATMENT CLINICS

(71) Applicants: Fresenius Medical Care Holdings, Inc., Waltham, MA (US); Fresenius Medical Care Deutschland GmbH, Bad Homburg (DE)

(72) Inventors: Martin Crnkovich, Walnut Creek, CA (US); Christian Schlaeper, Wehrheim (DE); Ashween Ganesh, Bay Point, CA (US); David Yuds, Hudson, NH (US)

(21) Appl. No.: 18/242,350

(22) Filed: Sep. 5, 2023

(51) Int. Cl. G16H 40/40; G16H 10/60; G16H 40/20; G16H 50/20

(52) U.S. Cl. CPC G16H 40/40; G16H 10/60; G16H 40/20; G16H 50/20

(57) ABSTRACT

Systems and methods are provided to incorporate a high intelligence vertical efficiencies (HIVE) scheduler into connected health systems, which interacts with various entities in the connected health systems, and utilizes intelligent algorithms and an array of sensors to optimize clinic operations. The HIVE scheduler optimizes clinic operations by dynamically adapting schedules across various tasks within the connected health system. The HIVE scheduler determines adjustment to the schedules based on information such as alert/alarm events from machines, a variety of sensor data from within and outside clinics, and historical patterns. The systems and methods are applicable to both hemodialysis (HD) and peritoneal dialysis (PD) applications.

### BACKGROUND

Medical devices are known for use in the treatment of renal disease. The two principal dialysis methods are hemodialysis (HD) and peritoneal dialysis (PD). During hemodialysis, a patient's blood that flows from an access site, is passed through a dialyzer of a dialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate, or dialysis solution. The membranous lining of the patient's peritoneum acts as a natural semi-permeable membrane that allows diffusion and osmosis exchanges to take place between the solution and the blood stream. Automated peritoneal dialysis machines, also called PD cyclers, are designed to control the entire peritoneal dialysis process so that it can be performed at home, usually overnight, without clinical staff in attendance. Both HD and PD machines may include displays with touch screens or other user interfaces that display information of a dialysis treatment and/or enable an operator or patient to interact with the machine.

Dialysis centers may experience a hum of activity with multiple pods of networked hemodialysis machines managed by teams of patient care technicians and nurses to get patients connected and treated in the most efficient manner possible. Staff coordinates care from the receptionist who greets the patients and checks them in to the technicians who weigh the patient, enter their data, and connect them, to the nurses who monitor their vitals and perform the rounds to administer medications and make critical adjustments and communicate with physicians.

A great deal of labor is involved in coordinating all these moving parts to a clinic which places a significant burden on clinic managers and staff. Everything from setting the staff schedule to ordering medications and administering them to performing individual blood pressure checks and foot checks takes time away from the kind of personal interaction with the patients that motivates them to be compliant in their own therapy. Additionally, this constant hum of activity strains staff and could cause them to miss out on subtle details in administering the standard of care.

### SUMMARY

In exemplary embodiment, the present application provides a connected health system, comprising one or more sensors configured to detect interruptions corresponding to a plurality of activities in the connected health system; one or more monitoring devices configured to receive information corresponding to a plurality of tasks; and a computing system. The computing system comprises: one or more processors; and a non-transitory computer-readable medium having processor-executable instructions stored thereon. The processor-executable instructions, when executed, facilitate: obtaining data from the one or more sensors, the data indicating the interruptions to the plurality of activities in the connected health system; determining, by analyzing the data from the one or more sensors, changes to existing schedules for the plurality of tasks; and sending alert/alarm events indicating the changes to the existing schedules for the plurality of tasks to the one or more monitoring devices.

In a further exemplary embodiment, the one or more sensors comprise one or more first sensors affiliated with a logistic company, the one or more first sensors are configured to monitor shipment information to the connected health system.

In a further exemplary embodiment, obtaining the data from the one or more sensors further comprises: obtaining, from the one or more first sensors, the shipment information. Determining the changes to the existing schedules for the plurality of tasks further comprises: determining, based on the shipment information, the changes to the existing schedules for the plurality of tasks.

In a further exemplary embodiment, the one or more sensors comprise one or more second sensors, the one or more second sensors are configured to tap into one or more social media data feeders to monitor local news.

In a further exemplary embodiment, a second sensor of the one or more second sensors is configured to monitor a social media data feeder of the one or more social media data feeders to monitor local water or power utilities.

In a further exemplary embodiment, the second sensor is further configured to transmit an event based on detecting interruptions to at least one of electricity and water supply.

In a further exemplary embodiment, a second sensor of the one or more second sensors is configured to monitor a social media data feeder of the one or more social media data feeders to monitor local traffic or weather news.

In a further exemplary embodiment, the second sensor is further configured to transmit an event based on detecting, from the social media data feeder, at least one of: disaster alerts; weather/storm tracking; and severe traffic conditions.

In a further exemplary embodiment, the one or more sensors comprise one or more third sensors, and wherein the one or more third sensors are configured to perform at least one of: monitoring patient checking in at a receptionist; monitoring dosing schedules; and monitoring patient feedback.

In a further exemplary embodiment, determining, by analyzing the data from the one or more sensors, the changes to the existing schedules for the plurality of tasks further comprises: sorting, based on the analysis of the data from the one or more sensors, priorities of the plurality of tasks; and determining, based on the priorities of the plurality of tasks, the changes to the existing schedules.

In another exemplary embodiment, the present application provides a connected health system, comprising: one or more machines configured to transmit one or more events associated with one or more tasks; one or more monitoring devices configured to receive information corresponding to a plurality of tasks comprising the one or more tasks; and a computing system. The computing system comprises: one or more processors; and a non-transitory computer-readable medium having processor-executable instructions stored thereon. The processor-executable instructions, when executed, facilitate: receiving the one or more events from the one or more machines; determining, based on the one or more events and existing schedules, priorities and assignments of the plurality of tasks; determining, based on the priorities and assignments of the plurality of tasks, updated schedules for the plurality of tasks; and sending the updated schedules to the one or more monitoring devices.

In further exemplary embodiment, each event of the one or more events comprises at least one of: an alarm; an alarm type; a severity level; and a number of response steps necessary based on the particular event.

In further exemplary embodiment, determining, based on the one or more events and existing schedules, the priorities and assignments of the plurality of tasks further comprises: sorting, based on the one or more events, priorities of the one or more machines; and determining, based on the priorities of the one or more machines, the priorities and assignments of the plurality of tasks.

In further exemplary embodiment, the processor-executable instructions, when executed, further facilitate: receiving information from a medical information system (MIS), the information indicating a test to be performed; and integrating the test to the existing schedules. The integrating the test to the existing schedules further comprises at least one of: integrating the test into an existing work flow; allotting extra time for the test; rescheduling a discussion with a dietician based on time being tight; blocking time for nurses and attending physician to discuss results of the test; updating billing information for insurance; and synchronizing the test with a monthly foot check based on that a nurse is available at the same time.

In a further exemplary embodiment, the processor-executable instructions, when executed, further facilitate: receiving information from a medical information system (MIS), the information indicating that a patient has been rerouted to another clinic; and rescheduling one or more tasks of the plurality of tasks corresponding to the patient.

In a further exemplary embodiment, the processor-executable instructions, when executed, further facilitate: monitoring, for each task of the plurality of tasks, progress of the respective task and a status of a respective clinician assigned to the respective task; generating, based on the monitoring results, metrics for the respective clinician corresponding to the respective task of the plurality of tasks; and determining recommended training for the respective clinician.

In yet another exemplary embodiment, the present application provides a connected health system, comprising: one or more monitoring devices configured to receive information corresponding to a plurality of tasks; and a computing system. The computing system comprises: one or more processors; and a non-transitory computer-readable medium having processor-executable instructions stored thereon. The processor-executable instructions, when executed, facilitate: determining, based on history data associated with completed tasks, one or more patterns; determining, based on the one or more pattern, changes to one or more tasks of the plurality of tasks; determining, based on the changes, updated schedules for one or more tasks of the plurality of tasks; and sending the updated schedules to the one or more monitoring devices.

In a further exemplary embodiment, the history data comprises at least one of: sensor readings; patient records; and machine operational metrics.

In a further exemplary embodiment, determining, based on the history data associated with the completed tasks, the one or more patterns further comprises: deriving the one or more patterns from the history data by applying a machine learning model.

In a further exemplary embodiment, determining, based on the one or more pattern, the changes to the one or more tasks of the plurality of tasks further comprises: correlating one or more events corresponding to the one or more tasks with the one or more patterns; and determining, based on the correlation results, the changes to the one or more tasks of the plurality of tasks.

### BRIEF DESCRIPTION OF THE DRAWINGS

FIG. 1 is a schematic diagram of an exemplary hemodialysis system having an optical blood monitoring system.

FIG. 2 is a block diagram of an exemplary network environment in a connected dialysis clinic.

FIG. 3 is a block diagram of one or more devices or systems within the exemplary environment of FIG. 2.

FIG. 4 is a block diagram demonstrating exemplary functionalities facilitated by a connected health system utilizing a HIVE scheduler.

FIG. 5 is a block diagram illustrating exemplary channels facilitating information exchange between a HIVE scheduler and users in a connected health system.

FIG. 6A is a flowchart of an exemplary process for performing task scheduling by a system.

FIG. 6B is a flowchart of an exemplary process for performing smart-auto-rescheduling by a system to dynamically update the schedules.

FIG. 7 is a flowchart of an exemplary process for coordinating activities by a system.

FIG. 8 is a flowchart of an exemplary process for performing advanced analysis and prediction by a system.

### DETAILED DESCRIPTION

Technological platforms exist for attempting to coordinate patient care in a clinic setting. However, these conventional technological platforms may experience inefficiencies because they may not be able to consider or correlate all necessary tasks, alarms and/or unexpected interventions, which can result in sub-optimal patient experience. Accordingly, there remains a technical need to improve upon and streamline the technological platforms used within treatment clinics to enhance both clinic efficiency and the quality of patient experiences.

Exemplary embodiments of the present application utilize connected health systems, smart algorithms, and an array of sensors to analyze and streamline clinic operations, resulting in an augmentation of the clinic's overall operational efficiency and a noteworthy alleviation of management burdens. For example, a high intelligence vertical efficiencies (HIVE) scheduler is integrated into the interconnected network, acting as a “central control” to orchestrate tasks among the various entities within the network. The HIVE scheduler engages with machines and devices (e.g., hemodialysis systems and sensors) in the network, utilizing control signals to facilitate data collection, process monitoring, and the scheduling of machine usage. Additionally, the HIVE scheduler engages with staff members and patients through data input/output (I/O) portals, collecting information encompassing staff and patient availability and/or preferences, thereby allowing the HIVE scheduler to finely tune schedules tailored to specific treatment processes, resulting in enhanced operational efficiency. In some variations, the HIVE scheduler is further linked to additional data sources (e.g., through sensors), including social media feeds. This integration empowers the HIVE scheduler to dynamically adjust existing schedules as needed. For instance, the HIVE scheduler may decide whether to cancel or rearrange scheduled treatments for specific patients on a particular day based on forecasts of adverse weather conditions.

The present disclosure describes an exemplary hemodialysis system and dialysis clinic as instances of a connected machine and a connected health system, respectively. It will be appreciated that the hemodialysis system and dialysis clinic disclosed hereinafter are merely exemplary. The principles discussed herein are also applicable to other types of network configurations, entities, and equipment.

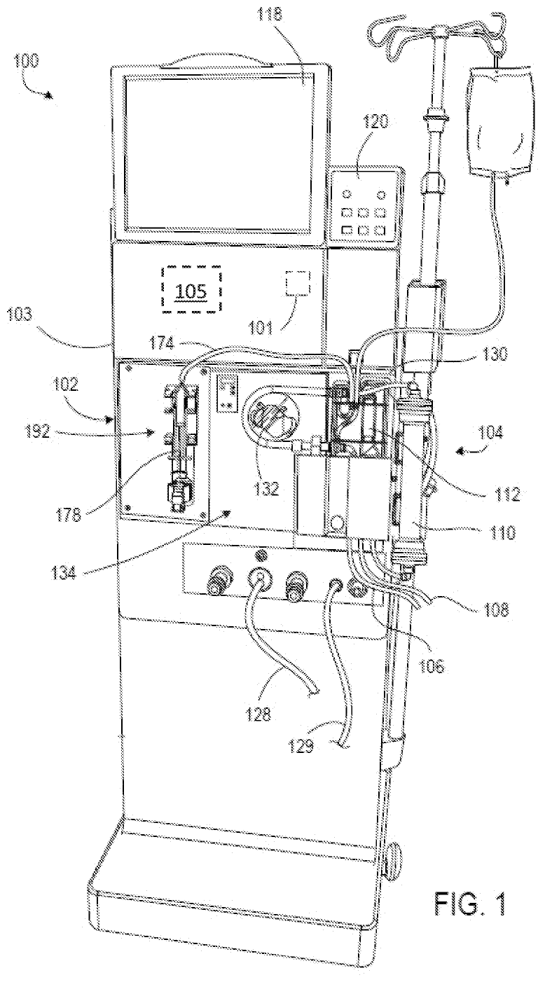

FIG. 1 shows an example implementation of a hemodialysis system 100. The hemodialysis system 100 includes a hemodialysis machine 102 connected to a disposable blood component set 104 that partially forms a blood circuit. During hemodialysis treatment, an operator connects arterial and venous patient lines 106, 108 of the blood component set 104 to a patient. The blood component set 104 may include an air management device 112 that may include, for example, a venous drip chamber.

The blood component set 104 is secured to a module 130 attached to the front of the hemodialysis machine 102. The module 130 includes the blood pump 132 capable of circulating blood through the blood circuit. The module 130 also includes various other instruments capable of monitoring the blood flowing through the blood circuit. The module 130 includes a door that when closed, as shown in FIG. 1, cooperates with the front face of the module 130 to form a compartment that is sized and shaped to receive the blood component set 104.

The blood pump 132 is part of a blood pump module 134. The blood pump module 134 includes a display window, a start/stop key, an up key, a down key, a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in a drip chamber.

The hemodialysis machine 102 further includes a dialysate circuit formed by the dialyzer 110, various other dialysate components, and dialysate lines connected to the hemodialysis machine 102. Many of these dialysate components and dialysate lines are inside the housing 103 of the hemodialysis machine 102 and are thus not visible in FIG. 1. During treatment, while the blood pump 132 circulates blood through the blood circuit, dialysate pumps (not shown) circulate dialysate through the dialysate circuit.

A drain line 128 and an ultrafiltration line 129 extend from the hemodialysis machine 102. The drain line 128 and the ultrafiltration line 129 are fluidly connected to the various dialysate components and dialysate lines inside the housing 103 of the hemodialysis machine 102 that form part of the dialysate circuit. During hemodialysis, the dialysate supply line carries fresh dialysate to the portion of the dialysate circuit located inside the hemodialysis machine 102. As noted above, the fresh dialysate is circulated through various dialysate lines and dialysate components, including the dialyzer 110, that form the dialysate circuit. As the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate and excess fluid drawn from the patient is carried to the drain via the ultrafiltration line 129.

The dialyzer 110 serves as a filter for the patient's blood. The dialysate passes through the dialyzer 110 along with the blood, as described above. A semi-permeable structure (e.g., a semi-permeable membrane and/or semi-permeable microtubes) within the dialyzer 110 separates blood and dialysate passing through the dialyzer 110. This arrangement allows the dialysate to collect toxins from the patient's blood. The filtered blood exiting the dialyzer 110 is returned to the patient. The dialysate exiting the dialyzer 110 includes toxins removed from the blood and is commonly referred to as “spent dialysate.” The spent dialysate is routed from the dialyzer 110 to a drain.

A drug pump 192 also extends from the front of the hemodialysis machine 102. The drug pump 192 is a syringe pump that includes a clamping mechanism configured to retain a syringe 178 of the blood component set 104. The drug pump 192 also includes a stepper motor configured to move the plunger of the syringe 178 along the axis of the syringe 178. A shaft of the stepper motor is secured to the plunger in a manner such that when the stepper motor is operated in a first direction, the shaft forces the plunger into the syringe, and when operated in a second direction, the shaft pulls the plunger out of the syringe 178. The drug pump 192 can thus be used to inject a liquid drug (e.g., heparin) from the syringe 178 into the blood circuit via a drug delivery line 174 during use, or to draw liquid from the blood circuit into the syringe 178 via the drug delivery line 174 during use.

The hemodialysis machine 102 includes a user interface with input devices such as a touch screen 118 and a control panel 120. The touch screen 118 and the control panel 120 allow the operator to input various different treatment parameters to the hemodialysis machine 102 and to otherwise control the hemodialysis machine 102. The touch screen 118 displays information to the operator of the hemodialysis system 100.

The hemodialysis machine 102 also includes a control unit 101 (e.g., a processor) configured to receive signals from and transmit signals to the touch screen 118 and the control panel 120. The control unit 101 can control the operating parameters of the hemodialysis machine 102, for example, based at least in part on the signals received by the touch screen 118 and the control panel 120. The hemodialysis machine 102 may also include a communication unit 105 that may be provided for wireless communication with a remote control device and may be communicatively coupled with the control unit 101.

The hemodialysis system depicted in FIG. 1 may be one of a plurality of hemodialysis systems in a dialysis clinic. Patients may come into the dialysis clinic for treatments at regular intervals, for example, on a Monday-Wednesday-Friday schedule or a Tuesday-Thursday-Saturday schedule.

It will be appreciated that the hemodialysis system depicted in FIG. 1 is merely exemplary. The principles discussed herein are applicable to other types of hemodialysis systems, as well as other types of dialysis systems and medical devices, including peritoneal dialysis systems.

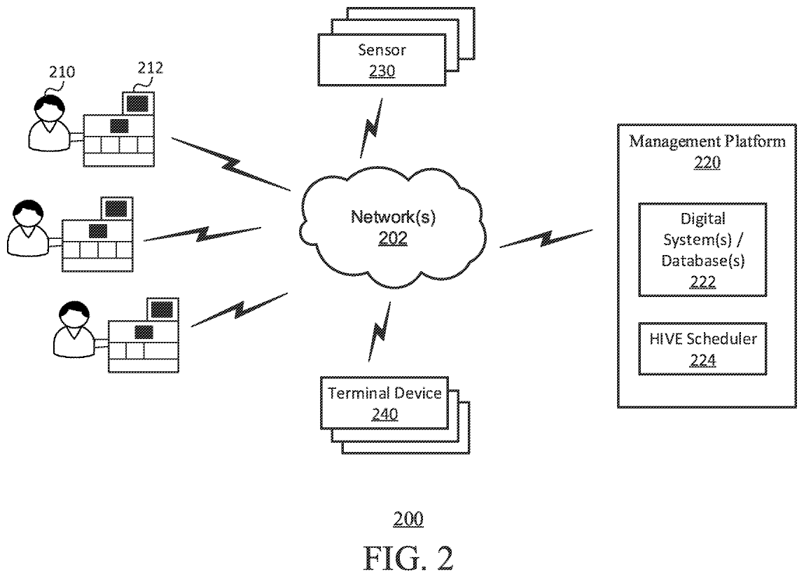

FIG. 2 is a block diagram of an exemplary network environment 200 in a connected dialysis clinic.

The network environment 200 includes one or more hemodialysis systems (e.g., the hemodialysis system depicted in FIG. 1) used to provide hemodialysis treatment to one or more patients (including a respective patient 211 and a respective hemodialysis system 212), an array of sensors 230, a plurality of terminal devices 240, and a management platform 220.

The entities in the network environment 200 communicate with each other via one or more networks 202, which may be wired, wireless, or both. By way of example, the network(s) 202 may include one or more Wide Area Networks (“WANs”), one or more Local Area Networks (“LANs”), one or more public networks such as the Internet, and/or one or more private networks. Wired connections may include Ethernet RJ-45 or fiber optic connections, and wireless connections may include Bluetooth or WiFi connections. For example, a display device or a sensor device of an optical blood monitoring system of each of the hemodialysis systems may include a communications interface and corresponding communications equipment for communicating with the management platform 220 via the wired or wireless connection. Similarly, the array of sensors 230 and the plurality of terminal devices 240 may include communications interfaces and corresponding communications equipment for communicating with the management platform 220 via the wired or wireless connection.

The array of sensors 230 may include a diverse assortment of sensing and detecting devices strategically positioned within the clinic, including temperature and humidity sensors, motion sensors, RFID detectors, and other relevant data acquisition tools like surveillance cameras. Additionally, the scope of the array of sensors 230 may encompass sensors beyond the clinic, extending to sensors affiliated with external departments or companies. For instance, certain devices within this sensor array may involve sensors from a logistics company, utilized to monitor shipments to the clinic. Furthermore, the array of sensors 230 may also integrate data feeders from diverse origins, including social media platforms. As an example, one of these sensors may tap into a social media data feed from local water or power utilities, while another sensor may access a social media data feed containing updates on local traffic or weather news. As such, the management platform 220 may obtain pertinent data from the array of sensors 230, which may influence present or upcoming schedule adjustments.

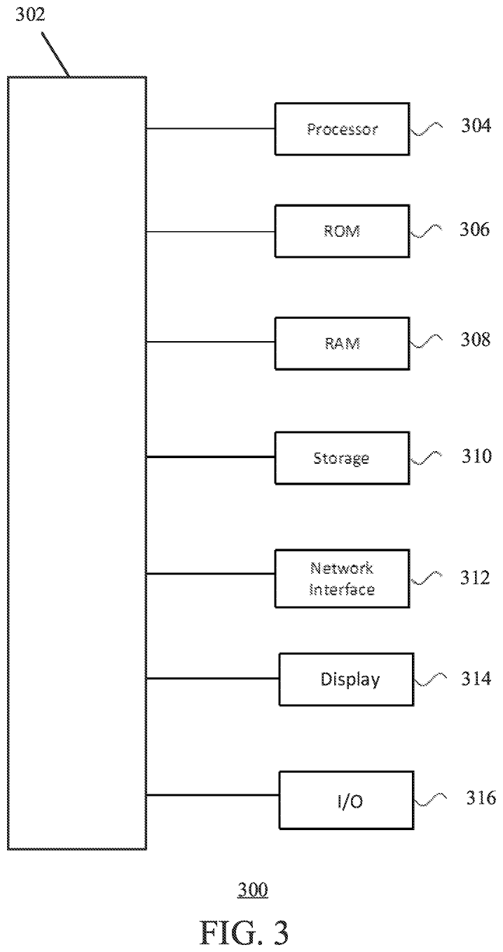

The terminal devices 240 act as data input/output (I/O) portals for both clinic staff and patients to provide and/or retrieve information from the clinic's health system (e.g., from the management platform 220). The terminal devices 240 may include at least some of the components, features, and functionality of the example computer system 300 of FIG. 3. By way of example and not limitation, a terminal device 240 may be embodied as a Personal Computer (“PC”), a laptop computer, a mobile device, a smartphone, a tablet computer, a virtual reality headset, a vehicle, a virtual machine, a handheld communications device, a vehicle computer system, an embedded system controller, a workstation, an edge device, any combination of these delineated devices, or any other suitable device.

The management platform 220 is not constrained to any particular hardware or software, and the management platform's configuration may be implemented by any kind of software applications, databases, and hardware infrastructure programming or hardware design—or a combination thereof. For instance, the management platform 220 may operate on a local computing system or server (including computing system(s)/device(s) as depicted in FIG. 3) within a dialysis clinic. Alternatively, the management platform 220 may function within a virtual machine (VM) hosted in a cloud environment, utilizing local or external servers to offer services and resources over the internet.

The management platform 220 communicates with the one or more hemodialysis systems, the array of sensors 230, and the terminal devices 240 to obtain pertinent information encompassing the hemodialysis systems, clinic environment, staff, patients, and other relevant factors.

In some examples, the management platform 220 may incorporate or be communicatively connected to one or more digital systems/databases 222 for managing patient health information. Examples of the digital systems/databases include but not limited to Electronic Health Record (EHR), Electronic Medical Record (EMR), and Medical Information System (MIS). An Electronic Medical Record (EMR) is a digital version of a patient's paper chart. It contains the medical and treatment history of a patient in a single healthcare provider's office, such as a doctor's practice or a clinic. EMRs focus on the clinical data and are primarily used for diagnosis and treatment within a specific healthcare organization. An Electronic Health Record (EHR) includes a broader range of patient information, including medical histories, diagnoses, allergies, medications, immunization records, lab results, and more. EHRs allow sharing of patient information across different healthcare providers, such as hospitals, specialists, pharmacies, and laboratories, ensuring coordinated and continuous care across the healthcare ecosystem. An MIS refers to a structured and organized digital platform that manages and facilitates the storage, retrieval, processing, and exchange of medical and healthcare-related information. The MIS is configured to collect, store, and manage data pertaining to patient health records, medical histories, diagnoses, treatments, prescriptions, lab results, and other relevant healthcare data. Medical information systems play a crucial role in enhancing healthcare delivery, improving patient care, enabling efficient clinical decision-making, and supporting administrative and operational functions within healthcare organizations.

The management platform 220 integrates a HIVE scheduler 224 that employs intelligent algorithms to efficiently align and manage the available resources within the management platform 220. An exemplary use case entails creating optimized schedules for hemodialysis treatments, encompassing tasks like scheduling patient appointments, configuring appropriate hemodialysis systems, and assigning staff members to tasks, all of which contribute to elevating clinic efficiency through strategic coordination of resources. The algorithms may include rule-based decision-making logic and/or trained machine learning (ML) models to facilitate decision-making processes.

In some variations, the management platform 220, incorporating a HIVE scheduler 224, may function across multiple interconnected clinics. This extends to scenarios where the management platform 220 orchestrates coordination among numerous dialysis clinics within a network or manages the coordination of diverse types of clinics within the same network.

It will be appreciated that the network environment depicted in FIG. 2 is merely exemplary. The principles discussed herein are also applicable to other types of connected health systems, network configurations, entities, and equipment.

FIG. 3 is a block diagram of an exemplary system and/or device 300 (e.g., a terminal device, a computing system, and/or a client/server device) within the environment 200. The device/system 300 includes a processor 304, such as a central processing unit (CPU), a graphic processing unit (GPU), controller, and/or logic, that executes computer executable instructions for performing the functions, processes, and/or methods described herein. In some examples, the computer executable instructions are locally stored and accessed from a non-transitory computer readable medium, such as storage 310, which may be a hard drive or flash drive. Read Only Memory (ROM) 306 includes computer executable instructions for initializing the processor 304, while the random-access memory (RAM) 308 is the main memory for loading and processing instructions executed by the processor 304. The network interface 312 may connect to a wired network or cellular network and to a local area network or wide area network. The device/system 300 may also include a bus 302 that connects the processor 304, ROM 306, RAM 308, storage 310, and/or the network interface 312.

In some examples, a display 314 may be integrated as part of the device/system 300 or may be provided as a separate device communicatively coupled to the device/system 300. The display may include a display device such as a Liquid Crystal Display (“LCD”), a Light Emitting Diode Display (“LED”), a plasma display, or any other type of display, and provide a Graphical User Interface (“GUI”) presented on the display for user input and data depiction.

The device/system 300 may also include various I/O devices 316 such as a keyboard, a mouse, a touchpad, a touch screen, a microphone, a camera, a biosensor, etc. A user (e.g., a staff member or a patient) may input data to the device/system 300 (e.g., a terminal device) through the I/O devices 316.

The components within the device/system 300 may use the bus 302 to communicate with each other. The components within the device/system 300 are merely exemplary and might not be inclusive of every component, server, device, computing platform, and/or computing apparatus within the device/system 300. Additionally, and/or alternatively, the device/system 300 may further include components that might not be included within every entity of environment 200.

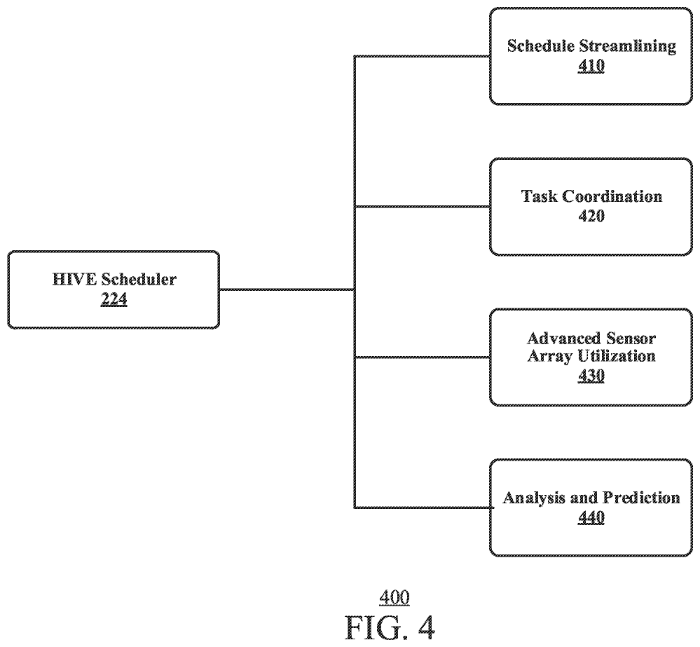

FIG. 4 is a block diagram 400 demonstrating exemplary functionalities facilitated by a connected health system utilizing a HIVE scheduler. As an example, the connected health system may be a connected dialysis clinic having a network environment configuration as depicted in FIG. 2. It will be appreciated that the principles discussed herein are applicable to other types of connected health systems, as well as other types of network environment configurations.

As described above, in a network environment 200 as shown in FIG. 2, the HIVE scheduler 224 obtains data from other entities within the network, for example, through the management platform 220. Then, the HIVE scheduler 224 applies smart algorithms to analyze the data and make decisions and/or predictions to more efficiently coordinate clinic operations.

Referring to block 410, the HIVE scheduler 224 can be utilized to facilitate schedule streamlining. A treatment schedule refers to a structured plan that outlines the timing, frequency, and specific interventions or therapies that a patient should receive as part of their medical treatment. It details the sequence and intervals at which various medical procedures, therapies, medications, or interventions are administered to a patient. Treatment schedules are essential in healthcare to ensure that patients receive the right treatments at the right times, optimizing the effectiveness of medical care and contributing to better health outcomes.

The HIVE scheduler 224 may achieve schedule streamlining through efficient data I/O and integration with the MIS. The following provides examples of using the HIVE scheduler 224 to streamline schedule for access flow measurement (AFM), medication administration, and other exemplary operations.

In one example, the HIVE scheduler 224 may obtain a patient schedule on the MIS and send messages/notifications to a corresponding machine/device to prompt a user (e.g., a nurse or physician) when to start a labor-intensive AFM. The HIVE scheduler 224 may analyze current schedules and decide to delay the AFM to better coordinate with alarms on that machine or other machines in the clinic or, when performing online hemodiafiltration (OL-HDF), to optimize the process. During the treatment, the HIVE scheduler 224 may monitor bibag bicarbonate levels and rates of usage using data collected from sensors. Based on this information, the HIVE schedule 224 may determine appropriate actions to be performed, such as triggering alarms or adjusting therapy parameters, to ensure effective and ongoing treatment.

In a further example, the HIVE schedule 224 may optimize online clearance (Kt/V) targets for the treatment. The ratio (Kt/V) compares the amount of fluid that passes through the dialyzer with the amount of fluid in the patient's body. To achieve the optimization, the HIVE scheduler 224 may determine when AFM needs to be performed to pick the right time to not impact reaching a critical Kt/V measurement. Additionally and/or alternatively, the HIVE scheduler 224 may choose to delay AFM until later to guarantee Kt/V target is reached or vice versa.

In an example of integrated medications schedules, the HIVE scheduler 224 may select a pre-planned window for medication administration, automatically taking into account how many patients need which medications and how long those medications take to prepare and administer. By linking the HIVE schedule 224 to staff and clinic calendars, the HIVE schedule 224 may manage caregiver schedules, freeing up the clinic manager for training activities and other personal interactions with the staff and patients.

In yet another example, the HIVE scheduler 224 may promptly address patient no-shows and reroute the patient to the next available slot at the clinic or even another nearby clinic.

Referring to block 420, the HIVE scheduler 224 can be utilized to facilitate task coordination. In the context of clinic operations, a task refers to a specific activity, assignment, or responsibility that needs to be accomplished as part of the daily functioning of the clinic. Tasks in a clinic can vary widely and encompass a range of activities, such as patient appointments, medical procedures, administrative duties, cleaning and maintenance, record-keeping, patient care, and more. Effective task management is crucial for ensuring the smooth and efficient operation of the clinic, providing quality patient care, and maintaining a well-organized healthcare environment.

Each task may be associated with a time tag. The HIVE scheduler 224 may distribute tasks across available caregivers to ensure that caregivers are not overwhelmed at specific intervals. For example, the HIVE scheduler 224 may prompt an assigned staff by sending an alert at the machine, a nurse screen, or the assigned staff member's mobile device to initiate prescribed task at specific patient. In some instances, the HIVE scheduler 224 may execute task coordination through collaboration with MIS. MIS recognizes tasks (such as initiating access flow during the early stages of treatment or administering specific medications later in the shift) for the shift as either assigned or prescribed. The HIVE scheduler 224 may fulfill the function of assigning or prescribing tasks for a specific shift.

Additionally, the HIVE scheduler 224 provides smart-auto-rescheduling to dynamically update the schedules. In one example, the HIVE schedule 224 may postpone tasks if other urgencies come up for an assigned staff. In that case, the HIVE scheduler 224 may recalculate and optimize timing for all remaining tasks. In another example, the management platform 220 (such as the MIS thereon) may continually provide reports about tasks completed and open tasks at any time and caregivers can pick and complete a task, which has been scheduled for later. In that case, the HIVE scheduler 224 may recalculate and optimize timing for all remaining tasks.

The smart-auto-rescheduling feature of the HIVE scheduler 224 proves particularly beneficial in situations requiring extensive coordination to avert delays that could negatively impact patients' satisfaction. For example, using a BCM (Body Composition Monitor) device requires a lot of coordination when a patient is not on a machine and must be seamlessly integrated into the patient check-in and preparation process. In other words, there are huge advantages to be gained if a system can coordinate the subtasks of laying the patient flat for two minutes, removing jewelry, socks and shoes, cleaning the measurement site, entering the patient data, and performing the measurement. In the busiest clinics when multiple patients need to get connected for dialysis in as little time as possible, any delays upset patients and schedules.

If a task requires a specific status of the dialysis machine, the HIVE scheduler 224 can contact a particular machine to get to this status (e.g. stable conductivity, completed system tests intra treatment, switch from online HDF to HD, etc.). Once the status is achieved by the machine, the HIVE scheduler 224 may prompt the caregiver to perform the next task. For instance, the prompt may take the form of a countdown clock showing the steps involved to prepare for the next task and when to initiate it. This form of prompt may save idle time of a caregiver waiting for a machine to get into a required status. Taking it a step further, this countdown and even the various task executions may be gamified to bring fun, excitement, and a sense of accomplishing a challenge to motivate staff to continually improve. Intra- and inter-clinic metrics can heighten the experience with competition and prizes.

Additionally and/or alternatively, the HIVE scheduler 224 may watch bandwidth consumption, monitor staff idling with various sensors (e.g., infrared sensors, respiration sensors, etc.), and gently nudge the staff with unexpected audio prompts like atypical alert sounds, music, etc. When there is expected downtime, the HIVE scheduler 224 may create fun distractions like broadcasting popular music to liven the mood and pick up the pace.

In some variations, the HIVE scheduler 224 may facilitate task coordination between connected clinics without necessitating labor-intensive efforts by receptionists to activate floating staff in the event of staff shortages. Further, the HIVE scheduler 224 may bring home dialysis systems into the mix by monitoring home dialysis operations to better plan if a home patient needs to come into the clinic for in-center treatment.

Referring to block 430, the HIVE scheduler 224 can be utilized to facilitate advanced sensor array utilization.

As demonstrated in FIG. 2, the network environment 200 may include an array of sensors 230, which may bring in information from non-dialysis machine devices to further enhance the awareness of the management platform 220 and specifically the HIVE scheduler 224.

In one example, the management platform 220 may link to a social media feed from the town's water or power utilities, so that the HIVE scheduler 224 may utilize the information to determine whether to prepare staff for critical interruptions in the schedule, such as electrical brownouts or additions of chlorine to the water supply that necessitates an increased water testing cadence at the clinic's RO system.

In another example, the management platform 220 may link to an array of sensors from a logistics company to track shipments to the clinic. As such, the HIVE scheduler 224 may use data from the sensors to plan draws for lab samples, monitor when medications, e.g., MICERA, are to be delivered to clinic, or any interruptions in supply chain.

In yet another example, the clinic may utilize near-field communications (NFC) technology (e.g., RFID) to sense which caregiver responds and the time needed for each response. The management platform 220 may collect data from the NFC devices and utilize the HIVE scheduler 224 to process this data to obtain valuable information, for example, to identify the most productive staff, to correlate it with outcomes from the machine's recorded treatment efficacy, and more. In this way, new metrics can be derived to demonstrate world-class care and to celebrate caregivers who personally achieve this.

In a further example, caregivers may use RFID wristbands or other wearable tags to check in with specific machines. For example, the RFID wristbands may provide a stream of data including user identification (ID), time, location, time to complete a specific task, actions taken, etc. The management platform 220 may obtain data through the sensing devices and use the HIVE scheduler 224 to analyze the data. For instance, the HIVE scheduler 224 may determine that three out of five nurses are occupied at machines, thus prompting the assignment of the task the fourth nurse. However, the HIVE scheduler 224 may also discern, based on other data source, if the fourth nurse is engaged in verbal interaction with a patient, potentially indicating their busyness. As such, the HIVE scheduler 224 may assign the task to the fifth nurse. Furthermore, the wristbands may be enhanced with motion sensors and/or heartrate sensors for additionally insights of clinicians, so that the HIVE scheduler 224 may adjust the assignments/tasks for the clinicians to rebalance their workload based on the data. In one example, the HIVE scheduler 224 may determine that a clinician is too idle, so the scheduler 224 may decide to remind the clinician to move to improve blood flow. In another example, the scheduler 224 may determine that a clinician is stressed, so the scheduler may determine to move the scheduled break for the clinician sooner. In this way, the HIVE scheduler 224 may rebalance workloads for the staff to reduce burnout.

Certain clinics mandate clinicians to inspect the patient's access at each machine every 15 minutes, aiming to reduce the risk of overlooking a leak that could cause harm and ensure early detection through scheduled rounds. Placing RFID tags on a clinician's foot, for example, may enable the HIVE scheduler 224 to more effectively differentiate between users and monitor their activities during surveillance.

Additionally, the management platform 220 may link to training resources on the Internet. The HIVE scheduler 224 on the platform 220 may analyze inefficiencies and/or deficiencies in the clinic to determine recommended training programs. Then, with the clinic manager's confirmation, the HIVE scheduler 224 may integrate training into staff schedules.

Referring to block 440, the HIVE scheduler 224 can be utilized to facilitate advanced analysis and prediction.

In an example, the HIVE scheduler 224 may continually monitor patient schedules and apply algorithms to identify trends. After algorithms start to note particular patients tend to get certain alarms within a certain time of starting, the system may present personalized tips to clinicians to highlight the underlying causes of these alarms. For instance, tracking which clinician put in the needles may uncover a pattern demonstrating more training is required for the particular staff. Alternatively, this information could yield insights in optimizing blood flow, dialysate flow, and UF rates.

In another example, the HIVE scheduler 224 may be combined with advanced devices/systems to enhance performance. For instance, a specific device may be connected in the health system, which can sense changes in hematocrit, categorizing it in one of three profiles: A, B, and C. The HIVE scheduler 224 may be utilized to track the profile trends of the device. If the profile is trending from B to C, the HIVE scheduler 224 may prompt staff to be ready to address an imminent crash and rebalance the clinic's resources to free clinicians to be pro-active rather than reactionary. This will have a remarkable effect on reducing nuisance alarms, making the clinic hum with quiet and peaceful efficiency.

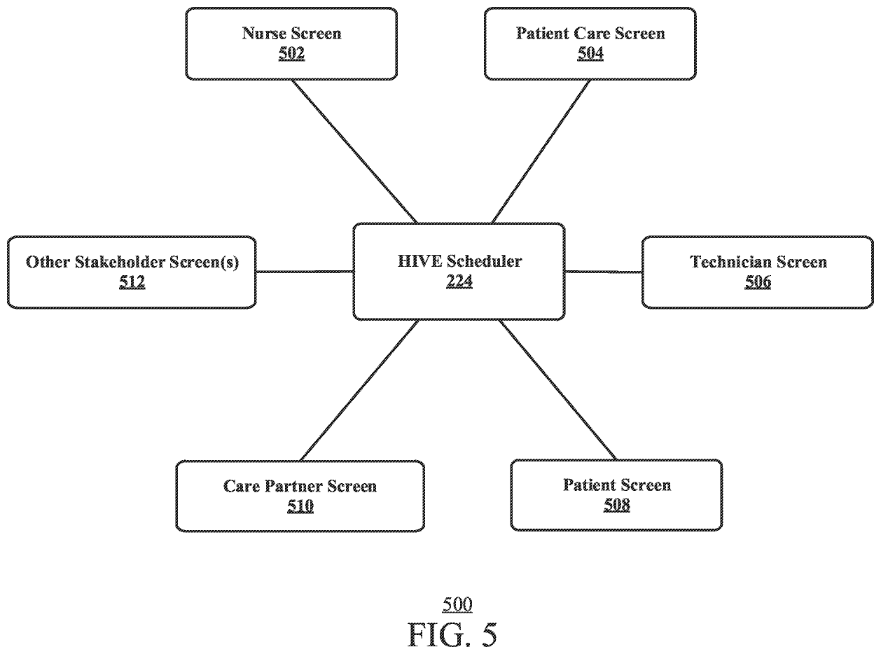

FIG. 5 is a block diagram 500 illustrating exemplary channels facilitating information exchange between the HIVE scheduler and users in a connected health system. As an example, these communication avenues could be offered by various entities within the network environment 200 depicted in FIG. 2. Moreover, these channels can be realized through diverse hardware or software designs, including integration of components like display 314 and I/O devices 316 as presented in FIG. 3, as well as through graphical user interfaces (GUI).

At block 502, a nurse screen may be accessible through mobile devices or on a centralized computer. The nurse screen may bring in all the information together from the connected health system and display an overall clinic summary to the head nurse and staff, which may include all nurse relevant patient dashboards. Presented information may include how UF Time, UF Rate, Dialysate Flow rate, Venous Pressure, TMP and treatment mode interact with one another. Through the nurse screen, nurses may be able to see all patient relevant details from the clinic in a single screen and be able to monitor patients who are currently in the clinic and their relevant parameters.

At block 504, a patient care screen may be accessible through mobile devices or on a centralized computer. The patient care screen may bring in all the information together from the connected health system(s) and display an overall summary to the staff, which may include all care giver relevant patient dashboards. Presented information may include tasks to assist patients in maintaining hygiene, mobility, and overall well-being. Through the patient care screen, patient care givers may be able to see patient relevant details from the clinic in a single screen and be able to monitor patients who are currently in the clinic and their needs.

At block 506, a technician screen may be accessible through mobile devices or on a centralized computer. The technician screen may bring in all the information together from the connected health system(s) and display an overall technical summary to the technical staff, such as biomedical equipment technicians or biomedical engineers (referred to as biomeds). Presented information may include the status of pumps, valves and sensors and how they interact with one another. Through the technician screen, technicians may be able to see machine relevant details from the clinic in a single screen and be able to monitor technical details. Technicians may schedule service pro-actively and coordinate via the care team scheduler to ensure minimal interruptions.

At block 508, a patient screen may be accessible through mobile devices. The patient screen may bring in all the information about how the patient is feeling during the treatment. Through the patient screen, patient feedback and smartphone parameters may be sent and monitored for improved scheduling of treatment. The HIVE scheduler 224 may instruct delivery of this critical information to the patient's social worker, which will help the social worker better coordinate with the rest of the care team.

At block 510, a care partner screen may be accessible through suitable systems/devices. Incorporating a care partner screen into the connected system may allow those who are not directly interacting with a specific machine (e.g., the dialysis machine) to provide a valuable stream of information to understand the peripheral impacts to the treatment. For instance, those supporting the patient (especially for home dialysis but also for in-center hemodialysis) can share information about their own energy levels, outlook, and ability to support the patient holistically—e.g., oftentimes despite the best efforts of a smoothly running dialysis unit, the patient's own diet works against him/her. Supporting the essential role of a care partner can help make critical connections to other dialysis staff like dieticians and social workers who can help improve the diet and reduce stress from treatment impediments for the patient. The care partner screen provides a different perspective than the patient screen to better reveal underlying issues that the patient may not even be aware of and would be particularly useful if the dialysis patient is showing cognitive decline. The HIVE scheduler 224 may instruct delivery of this critical information to the patient's social worker, which will help the social worker better coordinate with the rest of the care team.

At block 512, other stakeholder screen(s) may be accessible through suitable systems/devices. For instance, a stakeholder screen may be connected to a transportation (or logistic) company, through which the transportation company may update appropriate shipment information and/or be alerted of any delays in treatment resulting to later transport.

It will be appreciated that the channels and stakeholders demonstrated in FIG. 5 are merely exemplary. Additional stakeholders may be connected to the HIVE scheduler 224 through suitable stakeholder screen(s)/channel(s). Moreover, the principles discussed herein are also applicable to other types of connected health systems, channel configurations, and entities.

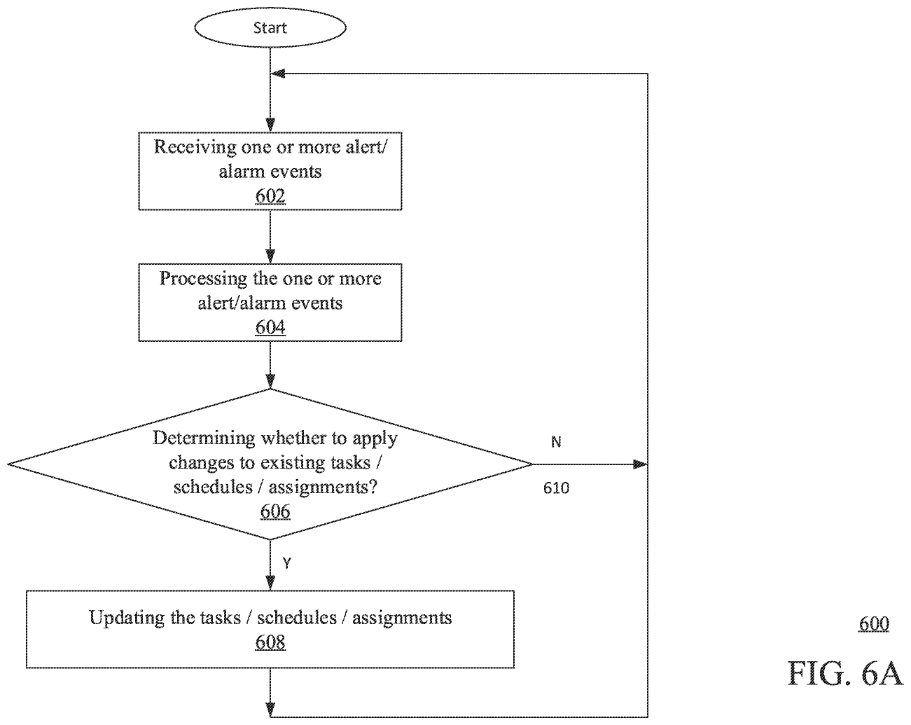

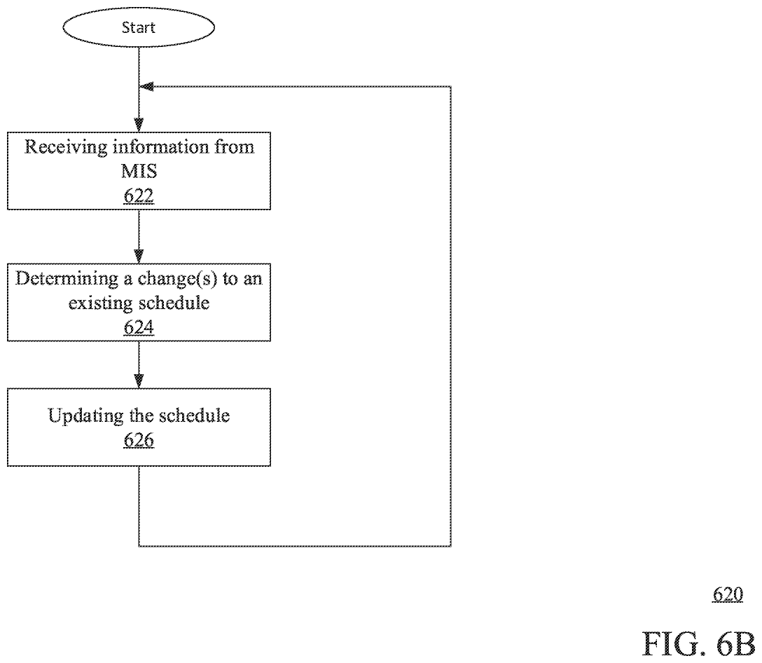

FIG. 6A is a flowchart of an exemplary process 600 for performing task scheduling by a system. For example, the system may be a connected health system as described in the network environment 200 in FIG. 2, or any suitable components (e.g., the management platform 220 or the HIVE scheduler 224) connected therein. However, it will be recognized that any of the following blocks may be performed in any suitable order and that the process 600 may be performed in any suitable environment and by any suitable system or platform.

At block 602, the system receives one or more alert/alarm events from one or more machines connected in the system. The alert/alarm events may be sent from one or more machines to one or more monitoring devices. The machines and/or the monitoring devices may be incorporated in or connected to the system. The system may utilize a HIVE scheduler 224 to process the alert/alarm events from the one or more machines to coordinate tasks and then relaying the outcomes to the one or more monitoring devices.

In one example, the one or more machines may broadcast alert/alarm events to of the one or more monitoring devices in the network 200. For example, the one or more machines may include hemodialysis systems 212, and the one or more monitoring devices may include terminal devices 240. In another example, the one or more machines may send alert/alarm events to specific monitoring devices that are relevant to particular events. A monitoring device may be a tablet, smartphone, smart watch, augmented reality/heads up display, etc., which may provide at least one of the communication channels as depicted in FIG. 5. As such, the one or more monitoring devices may provide information exchange portals to corresponding users (e.g., nurses, technicians, patients, care partners, etc.). An alert/alarm event may include various suitable information, including alarm, alarm type, severity level (high/medium/low), and response steps necessary.

In some examples, the system may be configured to receive alert/alarm events over a predefined time span, subsequently processing the accumulated events in batches. In some examples, the system may be configured to receive a specific count of alert/alarm events before initiating batch processing. In yet another examples, a hybrid approach could be employed, wherein the system combines both predetermined time intervals and a predefined event count to decide when to process the received events. However, it will be appreciated that these configurations are merely exemplary, and other suitable configurations may be applied.

At block 604, the system processes the one or more alert/alarm events. The system obtains information indicated by the alert/alarm events. For example, the system may obtain alarm, alarm type, severity level (high/medium/low), and response steps necessary based on the received event(s). The system uses the HIVE scheduler 224 integrated therein to coordinate a plurality of tasks based on the information from the event(s) and optionally additional information from other entities (e.g., data from sensors, machines, and terminal devices) connected in the health system.

The system may apply the HIVE scheduler 224 to sort priority between multiple machines, determine or learn the time required to respond, provide user task feedback, update task progress as an event is being addressed by any unique user, and schedule around concurrent and planned tasks. In some instances, the system informs corresponding users about alerts and how and when to respond for more efficient scheduling.

At block 606, the system determines whether to apply changes to existing tasks/schedules/assignments. Derived from the outcomes of block 604, the system may determine whether the received one or more events might cause changes to existing arrangements, such as previously arranged tasks/schedules/assignments. The system may obtain or establish these existing arrangements before the advent of the event or events currently undergoing processing.

The system may determine that there is a need to apply changes to existing arrangements in various scenarios. For instance, the system may determine that an event indicates that a new task has been generated, that a task progress has been updated, that an unexpected emergency has occurred (e.g., shortage of medication), or other suitable circumstances. In some examples, the changes may impact existing arrangements, possibly resulting in the cancellation of specific tasks or the rescheduling of existing tasks to different time frames. In some variations, the changes may not bear any influence on the pre-existing arrangements. This may involve the addition of new tasks into time slots that were previously unoccupied.

The system may determine that there is no need to apply changes to existing plans in various scenarios. For example, the system may deduce that the provided information, such as the alert/alarm events, pertains to machines and/or staff members not associated with any ongoing tasks. For example, an alert/alarm event may indicate a machine shutdown or the start/termination of a staff member's work shift.

At block 608, based on determining to apply changes to existing tasks/schedules/assignments, the system may update the tasks/schedules/assignments.

Otherwise, as indicated by branch 610, the system does not apply changes to the existing tasks/schedules/assignments.

In further examples, the system generates, based on monitoring of the plurality of tasks, metrics for specific users. In one example, the system knows the expected time to complete each task. As such, the system may monitor the progress of each task and the status of the clinician assigned to the respective task. Based on the obtained information, the system may generate metrics for the clinician corresponding to these tasks. In a further example, the system may highlight those clinician who may take longer to respond and then recommend applicable training by interfacing with training resources. In another example, the system may obtain data from RFID or other near field communications sensors to track clinicians as they interact with each machine and patient to ensure care goals are achieved.

Preventing Venous Needle Disconnect (VND) has attracted notable attention in specific clinics, prompting a desire for an effective means to realize routine monitoring. On the other hand, the system disclosed herein provides the task coordination capabilities that enhance monitoring of blood leak at the patients' access points, without the need for sensors directly on the patient access points. Specifically, the system can be used to seamlessly integrate staff rounds into all the other tasks and assign particular clinicians at particular times, thus providing complete coverage, and further the system can determine when and where there are gaps.

Furthermore, the system utilizes various sensing technologies to obtain a variety of information to help make decisions. In some instances, the system may obtain information from a combination of sensors to perform inductive reasoning. For instance, each dialysis machine may be equipped with a motion sensor. As such, the system may obtain paired information from dialysis machines and corresponding motion sensors to find out which clinicians are assigned to specific tasks and which clinicians are engaged in other tasks. This inductive reasoning by the system may be the foundational step towards initiating the tracking of team performance, thereby allowing more efficient workflows and continuous improvement in dialysis clinic staff. In some variations, unique staff IDs may be implemented for tracking of individual performance.

FIG. 6B is a flowchart of an exemplary process 620 for performing smart-auto-rescheduling to dynamically by a system to update the schedules. For example, the system may be a connected health system as described in the network environment 200 in FIG. 2, or any suitable components (e.g., the management platform 220 or the HIVE scheduler 224) connected therein. However, it will be recognized that any of the following blocks may be performed in any suitable order and that the process 620 may be performed in any suitable environment and by any suitable system or platform.

The system may incorporate a MIS on the platform 220, which collaborates with the HIVE scheduler 224.

At block 622, the system receives information from the MIS.

At block 624, the system determines a change(s) to an existing schedule based on the information from the MIS.

At block 626, the system updates the schedule.

In an example, the MIS may provide a note that the BCM test needs to be performed on a certain day at a certain time (in order for monthly measurements to be consistent). The HIVE scheduler 224 may automatically integrate that into an existing work flow, allotting extra time for the measurement, rescheduling a discussion with the dietician if time is tight, updating billing information for insurance, synchronizing the test with a monthly foot check if a nurse is available at the same time, etc.

If the patient has been rerouted to another clinic, the HIVE scheduler 224 can ensure this to seamless take place.

If the HIVE scheduler 224 records BP measurements or medication administration conducted for the same patient at that time, or even detecting via a microphone the patient speaking during the BCM test (the patient is to be silent), the HIVE scheduler 224 may intelligently invalidate the BCM test results in the MIS.

Additionally, the HIVE scheduler 224 may block more time for the nurse and attending physician to discuss the results of the BCM test by making adjustments to their calendars.

If the system determines the patient is retaining too much water based on BCM measurements, it could make additional recommendations like integrating CRIT-LINE monitoring during the dialysis treatment to better achieve the target dry weight and the HIVE scheduler 224 may coordinate with a dietician's schedule to visit with certain patients longer than others to improve compliance in fluid goals.

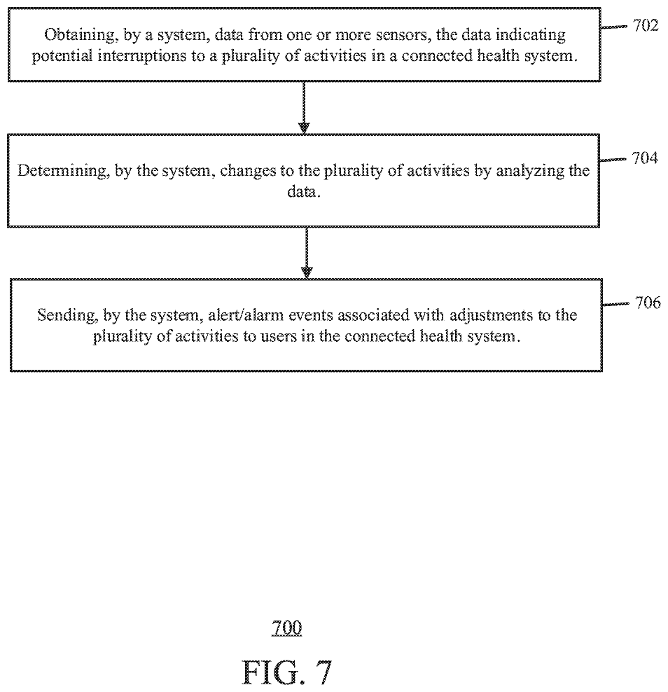

FIG. 7 is a flowchart of another exemplary process 700 for coordinating activities by a system. For example, the system may be a connected health system as described in the network environment 200 in FIG. 2, or any suitable components (e.g., the management platform 220 or the HIVE scheduler 224) connected therein. However, it will be recognized that any of the following blocks may be performed in any suitable order and that the process 700 may be performed in any suitable environment and by any suitable system or platform.

As described above with reference to FIG. 2, the network environment 200 may include an array of sensors 230, which may be of various types. For example, the sensors may take the form of linking to public utilities social media feeds to track the public water supply, disaster alerts, electricity interruptions (brownouts, high usage events), weather/storm tracking, etc. to intelligently coordinate clinic activities. Additionally, the array of sensors 230 may include sensors for patients checking in at the receptionist, medicine dosing schedules, product/supply deliveries, patient feedback (like schedules, preferences, or comfort level with particular staff members), and other various types of sensors.

At block 702, the system obtains data from one or more sensors, the data indicating potential interruptions to a plurality of activities in a connected health system.

At block 704, the system determines changes to the plurality of activities by analyzing the data.

At block 706, the system sends alert/alarm events associated with adjustments to the plurality of activities to users in the connected health system.

In one example, the system may obtain information from a sensor connected to a social media feed, indicating that the water utility is encountering runoff into a reservoir and subsequently issuing alerts to residents to boil water. Accordingly, the system may interpret this as a need to test water quality at the clinic's RO system more frequently until the condition clears to ensure water quality is maintained.

In another example, the system may obtain information from a sensor linked to a weather application, which may forecast a snowstorm. Consequently, the system may automatically rebalance workloads and staff schedules to schedule treatments for before and after the storm, taking an enormous burden off the clinic manager. In a further example, the system may coordinate with other local clinics to reroute patients if staffing levels are reaching critical levels, sending appointment notifications to patients to allow them to accept or decline the temporary shifts. It will be noted that the system may utilize task coordination capabilities to adapt dynamically to address other appropriate scenarios promptly.

In yet another example, the system may keep track of biomedical equipment technicians or biomedical engineers (referred to as biomeds) that are shared between multiple clinics. For example, the system may receive alerts through sensors indicating that the performance of certain components in a dialysis machine is decreasing before expected periodic maintenance. Upon receipt of these alerts, the system may automatically tag out the faulty machine, schedule a biomed for service, and select the next available machine to be prepared for use on the treatment floor even before the clinic opens for the day.

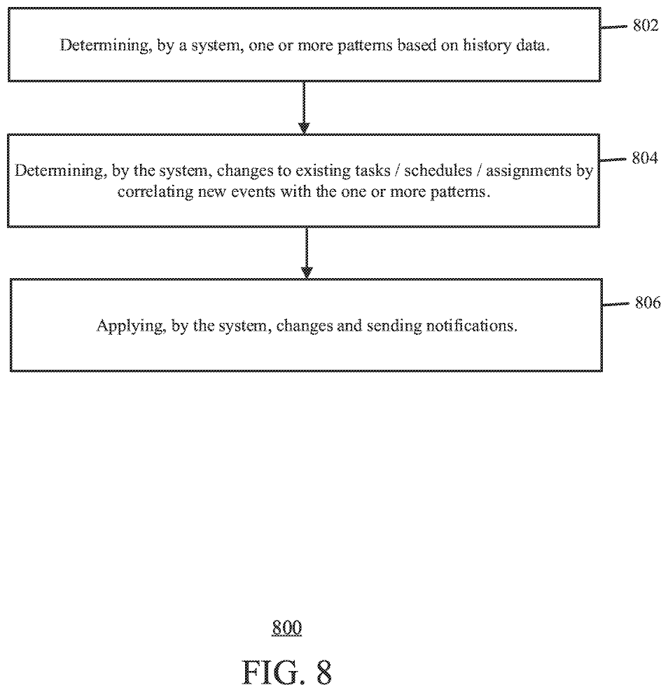

FIG. 8 is a flowchart of an exemplary process 800 for performing advanced analysis and prediction by a system. For example, the system may be a connected health system as described in the network environment 200 in FIG. 2, or any suitable components (e.g., the management platform 220 or the HIVE scheduler 224) connected therein. However, it will be recognized that any of the following blocks may be performed in any suitable order and that the process 800 may be performed in any suitable environment and by any suitable system or platform.

At block 802, the system determines one or more patterns based on history data. The system may store history data in a database. The historical data may be associated with accomplished tasks, which may encompass various information such as sensor readings, patient records, machine operational metrics, and more. The system may employ trained machine learning models (e.g., incorporated in the HIVE scheduler 224) to derive these patterns. Furthermore, the system may also utilize the historical data to train these machine learning models.

At block 804, the system determines changes to existing tasks/schedules/assignments by correlating new events with the one or more patterns.

At block 806, the system applies the changes and sends notifications.

In one example, the system may identify specific patients tending for higher rate of incidents or requiring more interventions. As such, the system may coordinate the pod and/or shift assignments to distribute the workload for nurses.

In another example, the system may note patient crashes (e.g., due to fluid drawn too fast as shown in hematocrit) through one or more sensors 230 (e.g., a CRIT-LINE blood monitor). Then the system may use the HIVE scheduler 224 can draw the correlation that this occurs more frequently when clinician X is monitoring the patient based on schedule. But the HIVE scheduler 224 may also discern that, based on RFID wristband sensors, Clinician Y who is unqualified is actually filling in. Rather than present this as a disciplinary action, the HIVE scheduler 224 may simply redirect Clinician Y to be at the other end of the clinic at some other task when this would normally occur. The HIVE scheduler 224 may also schedule training for Clinician Y to step up to the required level of competence all as a matter of course. This has the previously unattainable goal to effortlessly bring harmony to the dialysis clinic which in turn improves employee retention and reduces burnout in addition to raising the standard of care. Best of all, the clinic manager does not need to handle what could be a messy HR situation, reducing that workload and stress too.

It will be appreciated that although the exemplary embodiments discussed above include a dialysis clinic, the principles discussed herein are also applicable to other types of clinics, interconnected clinics, as well as other types of institutions/organizations.

It will be appreciated that the various machine-implemented operations described herein may occur via the execution, by one or more respective processors, of processor-executable instructions stored on a tangible, non-transitory computer-readable medium, such as a random access memory (RAM), read-only memory (ROM), programmable read-only memory (PROM), and/or another electronic memory mechanism. Thus, for example, operations performed by any device described herein may be carried out according to instructions stored on and/or applications installed on the device, and via software and/or hardware of the device.

All references, including publications, patent applications, and patents, cited herein are hereby incorporated by reference to the same extent as if each reference were individually and specifically indicated to be incorporated by reference and were set forth in its entirety herein.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present application covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A connected health system, comprising:
one or more sensors configured to detect interruptions corresponding to a plurality of activities in the connected health system;
one or more monitoring devices configured to receive information corresponding to a plurality of tasks; and
a computing system, comprising:
one or more processors; and
a non-transitory computer-readable medium having processor-executable instructions stored thereon, wherein the processor-executable instructions, when executed, facilitate:
obtaining data from the one or more sensors, the data indicating the interruptions to the plurality of activities in the connected health system;
determining, by analyzing the data from the one or more sensors, changes to existing schedules for the plurality of tasks; and
sending alert/alarm events indicating the changes to the existing schedules for the plurality of tasks to the one or more monitoring devices.

2. The system of claim 1, wherein the one or more sensors comprise one or more first sensors affiliated with a logistic company, wherein the one or more first sensors are configured to monitor shipment information to the connected health system.

3. The system of claim 2, wherein obtaining the data from the one or more sensors further comprises:
obtaining, from the one or more first sensors, the shipment information; and
wherein determining the changes to the existing schedules for the plurality of tasks further comprises:
determining, based on the shipment information, the changes to the existing schedules for the plurality of tasks.

4. The system of claim 1, wherein the one or more sensors comprise one or more second sensors, and wherein the one or more second sensors are configured to tap into one or more social media data feeders to monitor local news.

5. The system of claim 4, wherein a second sensor of the one or more second sensors is configured to monitor a social media data feeder of the one or more social media data feeders to monitor local water or power utilities.

6. The system of claim 5, wherein the second sensor is further configured to transmit an event based on detecting interruptions to at least one of electricity and water supply.

7. The system of claim 4, wherein a second sensor of the one or more second sensors is configured to monitor a social media data feeder of the one or more social media data feeders to monitor local traffic or weather news.

8. The system of claim 7, wherein the second sensor is further configured to transmit an event based on detecting, from the social media data feeder, at least one of:
disaster alerts;
weather/storm tracking; and
severe traffic conditions.

9. The system of claim 1, wherein the one or more sensors comprise one or more third sensors, and wherein the one or more third sensors are configured to perform at least one of:
monitoring patient checking in at a receptionist;
monitoring dosing schedules; and
monitoring patient feedback.

10. The system of claim 1, wherein determining, by analyzing the data from the one or more sensors, the changes to the existing schedules for the plurality of tasks further comprises:
sorting, based on the analysis of the data from the one or more sensors, priorities of the plurality of tasks; and
determining, based on the priorities of the plurality of tasks, the changes to the existing schedules.

11. A connected health system, comprising:
one or more machines configured to transmit one or more events associated with one or more tasks;
one or more monitoring devices configured to receive information corresponding to a plurality of tasks comprising the one or more tasks; and
a computing system, comprising:
one or more processors; and
a non-transitory computer-readable medium having processor-executable instructions stored thereon, wherein the processor-executable instructions, when executed, facilitate:
receiving the one or more events from the one or more machines;
determining, based on the one or more events and existing schedules, priorities and assignments of the plurality of tasks;
determining, based on the priorities and assignments of the plurality of tasks, updated schedules for the plurality of tasks; and
sending the updated schedules to the one or more monitoring devices.

12. The system of claim 11, wherein each event of the one or more events comprises at least one of:
an alarm;
an alarm type;
a severity level; and
a number of response steps necessary based on the particular event.

13. The system of claim 11, wherein determining, based on the one or more events and existing schedules, the priorities and assignments of the plurality of tasks further comprises:
sorting, based on the one or more events, priorities of the one or more machines; and
determining, based on the priorities of the one or more machines, the priorities and assignments of the plurality of tasks.

14. The system of claim 11, wherein the processor-executable instructions, when executed, further facilitate:
receiving information from a medical information system (MIS), the information indicating a test to be performed; and
integrating the test to the existing schedules, wherein the integrating the test to the existing schedules further comprises at least one of:
integrating the test into an existing work flow;
allotting extra time for the test;
rescheduling a discussion with a dietician based on time being tight;
blocking time for nurses and attending physician to discuss results of the test;
updating billing information for insurance; and
synchronizing the test with a monthly foot check based on that a nurse is available at the same time.

15. The system of claim 11, wherein the processor-executable instructions, when executed, further facilitate:
receiving information from a medical information system (MIS), the information indicating that a patient has been rerouted to another clinic; and
rescheduling one or more tasks of the plurality of tasks corresponding to the patient.

16. The system of claim 11, wherein the processor-executable instructions, when executed, further facilitate:
monitoring, for each task of the plurality of tasks, progress of the respective task and a status of a respective clinician assigned to the respective task;
generating, based on the monitoring results, metrics for the respective clinician corresponding to the respective task of the plurality of tasks; and
determining recommended training for the respective clinician.

17. A connected health system, comprising:
one or more monitoring devices configured to receive information corresponding to a plurality of tasks; and
a computing system, comprising:
one or more processors; and
a non-transitory computer-readable medium having processor-executable instructions stored thereon, wherein the processor-executable instructions, when executed, facilitate:
determining, based on history data associated with completed tasks, one or more patterns;
determining, based on the one or more pattern, changes to one or more tasks of the plurality of tasks;
determining, based on the changes, updated schedules for one or more tasks of the plurality of tasks; and
sending the updated schedules to the one or more monitoring devices.

18. The system of claim 17, wherein the history data comprises at least one of:
sensor readings;
patient records; and
machine operational metrics.

19. The system of claim 17, wherein determining, based on the history data associated with the completed tasks, the one or more patterns further comprises:
deriving the one or more patterns from the history data by applying a machine learning model.

20. The system of claim 17, wherein determining, based on the one or more pattern, the changes to the one or more tasks of the plurality of tasks further comprises:
correlating one or more events corresponding to the one or more tasks with the one or more patterns; and
determining, based on the correlation results, the changes to the one or more tasks of the plurality of tasks.
